(19) United States
(12) Patent Application Publication
(10) Pub. No.: US 2011/0098732 A1
(43) Pub. Date: Apr. 28, 2011

(54) VISCERAL TISSUE APPROXIMATING METHOD AND DEVICE

(76) Inventor: MOISES JACOBS, MIAMI, FL (US)

(21) Appl. No.: 12/913,476

(22) Filed: Oct. 27, 2010

Related U.S. Application Data
(60) Provisional application No. 61/255,430, filed on Oct. 27, 2009.

(51) Int. Cl. A61B 17/11

(52) U.S. Cl. 606/153

(57) ABSTRACT

The present invention is referred to a tissue approximating device comprising a two-piece anastomosis clamp that can be used to approximate two tissue sections together via open or laparoscopic technique, via Natural Orifice Translumenal Endoscopic Surgery (NOTES) or via single site surgery. The clamp includes a first member and a second member, where the clamp members are operable configured to fasten together, in juxtaposition to establish an anastomosis. The first clamp member includes a set of spikes and with a circumferential indentation and the second clamp member includes also a set of complementary receptacles for receiving said spikes and a circumferential indentation too. Said members have a circular shape, and they are made out of an absorbable material. The purposed method includes the steps of:

a) Cutting the bowel for removing a portion thereof, thus defining two loose ends of bowel tissue;
b) Inserting into one loose end of bowel tissue a first annular bio-absorbable clamp member, including a set of spikes on the front face,
c) Inserting into the second loose end of bowel a second annular clamp member including a stent-like tube integral therewith and set of complementary receptacles aligned with said spikes and on its front face;
d) Anchoring said first clamp members to the bowel's tissue;
e) Introducing said first clamp member around said stent-like tube also made of bio-absorbable material;
f) Approximating both clamp members by moving said first clamp member towards said second clamp member around the stent-like tube until the first annular member remains in front of the second annular clamp member;
g) Inserting said spikes into said receptacles, and
h) Approximating the end edges of said loose ends of the bowel if necessary.

### CROSS-REFERENCE TO RELATED APPLICATION

This application claims the benefit of co-pending U.S. Provisional Patent Application Ser. No. 61/255,430, filed on Oct. 27, 2009, which is incorporated herein in its entirety.

### BACKGROUND OF THE INVENTION

1. Field of the Invention

The present invention relates, in general, to surgical devices and more particularly is referred to a visceral tissue approximating device comprising a two-piece anastomosis clamp that can be used to approximate two tissue sections together via an open or laparoscopic technique, via Natural Orifice Translumenal Endoscopic Surgery (NOTES) or via single site surgery. Even more particularly the present invention is referred to a visceral tissue approximating method and device especially useful for approximating and anastomosing visceral tissue such as, for example, bowel tissue.

2. Description of the Prior Art

Many surgical procedures involve a suturing maneuver. Suturing of body tissues is a time consuming aspect of most surgical procedures. Modern surgical procedures tend to avoid performing a large opening to expose the area. Instead, performing small incisions to introduce endoscopes or laparoscopes is preferred in conjunction with specialized surgical instrumentation to detect, diagnose, and repair areas.

Laparoscopic surgery, also called minimally invasive surgery (MIS), is a modern surgical technique in which operations are performed through small incisions (usually 0.5-1.5 cm) as compared to larger incisions needed in traditional surgical procedures.

There are a number of advantages to the patient with laparoscopic surgery versus an open procedure. These include:

less post operative discomfort since the incisions are much smaller;

quicker recovery times;

shorter hospital stays;

earlier return to full activities;

much smaller scars;

less internal scarring;

reduced pain from infection and hemorrhaging, between others.

Access to the operative site using minimally invasive techniques is accomplished by inserting small tubes called ports into a body cavity. These tubes have a diameter of, for example, half an inch. There are several instruments in the prior art for suturing within a body cavity through these port tubes like the one described by Mulhollan et al. in the U.S. Pat. No. 4,621,640.

Some common examples of laparoscopic surgeries are: gallbladder removal, hernia repair, appendix removal, tubal ligation, etc. Depending on the type of surgery to be performed is the part of the body in which the incision may be made.

Natural orifice translumenal endoscopic surgery (NOTES) is a surgical technique whereby scarless abdominal operations can be performed with an endoscope passed through a natural orifice (mouth, urethra, anus, etc.) then through an internal incision in the stomach, vagina, bladder or colon, thus avoiding any external incisions or scars. Natural orifice translumenal endoscopic surgery generally requires no incisions because instruments, such as staple guns, can be inserted through the mouth and snaked down the esophagus. If the work to be done involves, for example, the lower portion of the intestines, the instruments can be inserted through the rectum. Another procedure known as transvaginal approach for the placement of the sutures has found favor among physicians. For example, a nephrectomy may be performed with a transvaginal retroperitoneal NOTES technique. Nephrectomy performed through a natural orifice could minimize postoperative recovery. The vagina has been considered a viable route for kidney retrieval also following laparoscopic nephrectomies.

Hence, alternatives have been sought to use minimally invasive surgery which would include laparoscopic surgery, or the new approach called “mini-incision surgery” using the principles of traditional open surgery, along with some of the equipment advances of laparoscopy.

In all the above cited procedures, an anastomosis is involved. Anastomosis is a surgical connection between two structures. It usually means a connection that is created between tubular structures, such as blood vessels or loops of an intestine. For example, when part of an intestine is surgically removed, the two remaining ends are sewn or stapled together (anastomosed), and the procedure is referred to as an intestinal anastomosis. There are several surgical procedures in which are necessary to perform an anastomosis, and even though there are several different types of devices and techniques for doing that, a clamp is one of them.

There are particular cases of anastomoses that pose special threads or risks to the patient like the anastomoses of the bowel. One of the risky situations in which the life of the patient is in risk relates to the possibility of the leakage of internal fluids into the abdominal cavity. In accordance with different statistics, between 3-15% of the bowel anastomosis procedures presents a leakage, and every leakage means the life of the patient is in great risk.

For example, in cases in which a section of the bowel must be removed, once the cut-off procedure is completed, the loose ends of the bowel must be re-connected or linked. This is a very well known procedure usually involving clipping the ends of each portion of the bowel. The problem resides on the fact that the gastric and intestinal fluids start flowing through the wound and if any deficiency in the anastomosis is found, said fluids may go through the wound to the abdominal cavity, creating a life-threatening situation for the patient.

There are several tissue approximating devices and surgical staplers for performing the anastomosis in the prior art. For example, U.S. Patent Application Serial No. 20080114383 filed by Hunt et al. provides a two-piece anastomosis fastener that can be used to join two tissue sections together in accordance with Natural Orifice Transendoscopic Surgery (NOTES). The fastener may be releasably attached to a fastener applying instrument for delivery in accordance with such procedures. The fastener includes a first member and a second member, where the clamp members are operably configured to fasten together to clamp and hold tissue, such as gastric tissue, in juxtaposition to establish an anastomosis. The first clamp member and the second clamp member are coupled with an adhesive.

U.S. Pat. No. 7,033,370 granted to Gordon et al. teaches about a method and device for the placement of sutures and for the purpose of approximating tissue. The invention relates to devices for approximation, ligation and fixation of tissue using a suture, to various constituent parts comprising said devices, and particularly to the placement of sutures into certain difficult to access ligamental structures, to the approximation of tissue separated by an endosurgical trocar being inserted into a body cavity, and to approximation, ligation, and fixation of body tissue using both traditional open surgical and endosurgical techniques and instruments. Loading of suture material including needles into the device is also included, and the introduction and placement of the device into the body cavity, with the distal end having deployable needle guides, extending the needle guides either simultaneously or individually to the periphery of the wound, engaging the wound with the needle guides, driving the needles and suture material through the tissue to be approximated into a catch mechanism, retracting the needle guides and withdrawing the device, leaving a loop of suture material in the margin of tissue. The suture may then be tied to approximate the wound and excess suture material cut off.

U.S. Pat. No. 6,997,932 granted to Dreyfuss et al. shows a device for the placement of sutures and for approximating tissue includes an elongate body member, a suture deployment system, and a catch. Methods of placing sutures include inserting and deploying in a patient a device including an elongate body member, a suture deployment system, and a catch are also encompassed. The suture deployment system is disposed at a distal portion of the elongate body member, and includes a suture carrier having a sharpened distal end for tissue penetration and a notch for holding a formed suture tip. The catch is disposed on the elongate body member to receive and retain the formed suture tip.

Also U.S. Pat. No. 5,735,445 granted to Vidal et al. teaches about a surgical stapler having a supporting frame, replaceable staple cartridge, an anvil, a mechanism for approximating the cartridge relative to the anvil, and a mechanism for firing the device to crimp the staples against the anvil in a manner to enable the surgeon to substantially simultaneously place one or more rows of surgical staples in organs or tissue. The device, while at all times permitting approximation of the cartridge relative to the anvil, provides a lockout feature for preventing retiring if the staple cartridge is spent. In this way, the device can be used as a clamping mechanism even after the staples have been fired.

U.S. Pat. No. 5,678,748 granted to Plyley et al. is referred to a surgical stapler for use by a surgeon to place one or more rows of surgical staples in organs or tissue. The stapler is of a character having a supporting frame, a replaceable staple cartridge, an anvil, a mechanism for approximating the cartridge relative to the anvil, and a mechanism for firing the device to crimp the staples against the anvil. The device, while at all times permitting approximation of the cartridge relative to the anvil, provides a novel safety mechanism that clearly indicates to the surgeon that the staples have been fired from the staple cartridge and simultaneously disables the device until it is manually reset.

Finally, U.S. Pat. No. 6,926,724 describes temporary biocompatible stent and method for visceral anastomosis. The stent is provided with an integral means for maintaining the structural stability of the stent while providing substantial flexibility. The method comprises fitting the luminal stumps of the viscus over either end of a stent of the invention, and joining the ends of each stump together. A short time after completion of the anastomosis, the stent dissolves and is absorbed safely into the body. The stent and method can be beneficially used in laparoscopic or more invasive traditional surgical procedures. The stent and method are particularly well suited for anastomosis of the bowel.

None of the above described devices or methods involve a safe and practical device and method through which the bowel anastomosis procedure may be performed providing to the surgeon and the patient the peace of mind that no leakage of gastric and intestinal fluids to the abdominal cavity will occur. As such, a tissue approximating method and device for performing the anastomosis of visceral tissue (like, for example, the bowel) through a fast and simple procedure is still desired in the market.

### SUMMARY OF THE INVENTION

A main object of the present invention is to provide a tissue approximating device comprising a two-piece anastomosis clamp that can be used to approximate two tissue sections together via an open or laparascopic technique, via Natural Orifice Transendoscopic Surgery (NOTES) or via single site surgery.

It is another object of the present invention to provide a clamp device for performing the anastomosis comprising a two-piece circular anastomosis clamp, the first piece including a set of spikes and the second piece including a stent-like tube integral therewith and a set of complementary orifices in which said spikes can be fitted in.

Yet another object of the present invention is to provide a tissue approximating device made of absorbable or biodegradable material. This device can be absorbed by the body several weeks of the month after the procedure without the necessity of performing a new surgical procedure to take it out of the patient.

Another object of the present invention is to provide a biodegradable and absorbable stent-like tube capable of avoiding fluid leakage and the risks involved therewith.

Yet another object of the present invention is to provide a method for performing a bowel anastomosis, including the insertion of a bio-absorbable disc-shaped clamp member integral with a stent-like tube creating two independent simultaneous actions: the bypass of the gastric fluids through the stent-like tube (avoiding the direct contact between the gastric fluids and the wound) and the safe and precise joint of the bowel tissue through the use of said disk-like approximating device.

Also another aspect of the purposed invention comprises a device which can be used for primary anastomosis, or, if delivered via an endoscope, colonoscope, or a bougie like delivery mechanism, it can also be used to protect an anastomosis created by other means (sutures, staples, glue, etc.).

In summary, the present invention is referred to a tissue approximating device and method comprising a two-piece anastomosis clamp that can be used to approximate two tissue sections together via an open technique, via Natural Orifice Translumenal Endoscopic Surgery (NOTES) or via single site surgery and a stent-like bio-absorbable tube which is integral with one of said clamps, for bypassing the gastric fluids avoiding the direct contact of the gastric fluids and the wound after the procedure. The clamp member includes a first circular member and a second circular member, where the diameter of each disc must coincide with the internal diameter of the bowel. Clamp members are operable configured to fasten together to clamp and hold between each other, in juxtaposition to establish an anastomosis. The first circular clamp member includes a set of spikes and the second clamp member includes a stent-like tube integral therewith and a set of receptacles for receiving said spikes. Each of said clamps includes next to its outer edge a set of indentations through which the surgeon can tie up said circular clamp to the bowel. Said clamp members may have different diameters depending on the size of the bowel to be anastomosed, and they are made of an absorbable material. During the surgical procedure, said second clamp member and the tube integral therewith (also made of an absorbable or biodegradable material) will be inserted into the bowel and said first and second clamp members are approximated to define the anastomosis. In addition, said biodegradable or absorbable stent-like tube in inserted into the bowel to create a bypass of gastric fluids over the anastomosed portion of the bowel.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is an exploded perspective view of a preferred embodiment of the tissue approximating device of the present invention. The clamp members define a circular shape for performing a one-shot anastomosis of a cylindrical organ like a bowel. It can also be seen that one of the clamp members includes a protective tube integral thereof.

FIGS. 2A-2E are a sequence of cross-sectional views illustrating the purposed method step by step, starting with the removal of a portion of the bowel until the final reconnection of the loose ends of said bowel using the purposed device and method of the present invention.

FIG. 3 is a cross sectional view of the anastomosed bowel once the procedure in accordance with the present invention was completed; finally:

FIG. 4 is a cross sectional view of the anastomosed bowel once the wound healed, and the different elements used during the procedure have been absorbed by the patient's body.

### DETAILED DESCRIPTION OF REPRESENTATIVE EMBODIMENTS

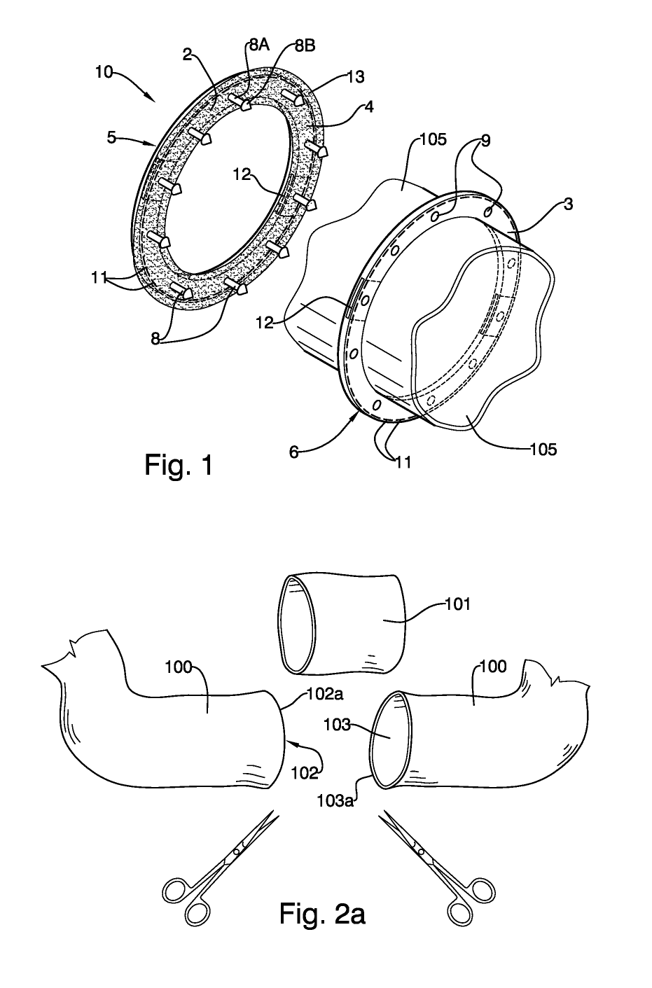

Referring now to the first preferred embodiment of the present invention, illustrated in FIG. 1, the invention is directed to a circular tissue approximating device 10 comprising a circular two-piece anastomosis clamp that can be used to approximate two tissue sections together. The clamp includes a first annular member 2 and a second annular member 3, where the clamp members are operable configured to fasten together to clamp, in juxtaposition to establish an anastomosis.

Said first annular clamp member 2 includes a front face 4 and a rear face 5. On said front face 4 a set of perpendicularly-arranged, equally-spaced arranged in a circle spikes 8 are included. Each spike presents a shape similar to an arrow, with a rectilinear thin body 8A and a V-shaped or blunt rounded, or cone shaped tip 8B at its end.

Said second annular clamp member 3 in turn comprises an annular body with a front face 7 and a rear face 6. A set of passing through equally-spaced orifices 9 are included on said faces 6-7. Said orifices have a complementary arrangement with the above cited spikes 8, so each spike 8 of the first member 2 can fit in each orifice 9 of the second member 3. Integral to said clamp member 3 a protective tube 105 is included. Both parts are molded together and define a sole integral piece.

Furthermore, in each clamp member 2-3 a set of regularly spaced indentations are included, which are used to secure each annular member 2-3 to the bowel as will be explained in detail below.

Furthermore, each spike 8 presents an arrow-like shape with a central linear body 8a and a V-shaped or blunt rounded, or cone shaped tip 8b. The reason behind this form is related to the purpose of this clamp device. Once the clamp is fired, the spike 8 will dig into the small orifices 9.

FIG. 1 illustrates the first embodiment of the purposed invention. This is an exploded perspective showing both members in a general perspective view. Both members are separated and they are in the position they will have before the clamp is fired. Even though it is not illustrated in the present set of drawings, these first and second members 2-3 will be loaded in a clamp firing device during the surgical procedure, which in turn may be fired endoscopically or laparoscopically. Also said clamps members 2-3 may be jointed manually if the surgeon has an open surgical field.

It is important to point out that said clamp member 3 defines an absorbable or biodegradable integral piece with the protective tube made of the same material 105. As will be explained below, both parts are molded together and define a sole piece. They are introduced in the bowel together defining the main part of the purposed invention.

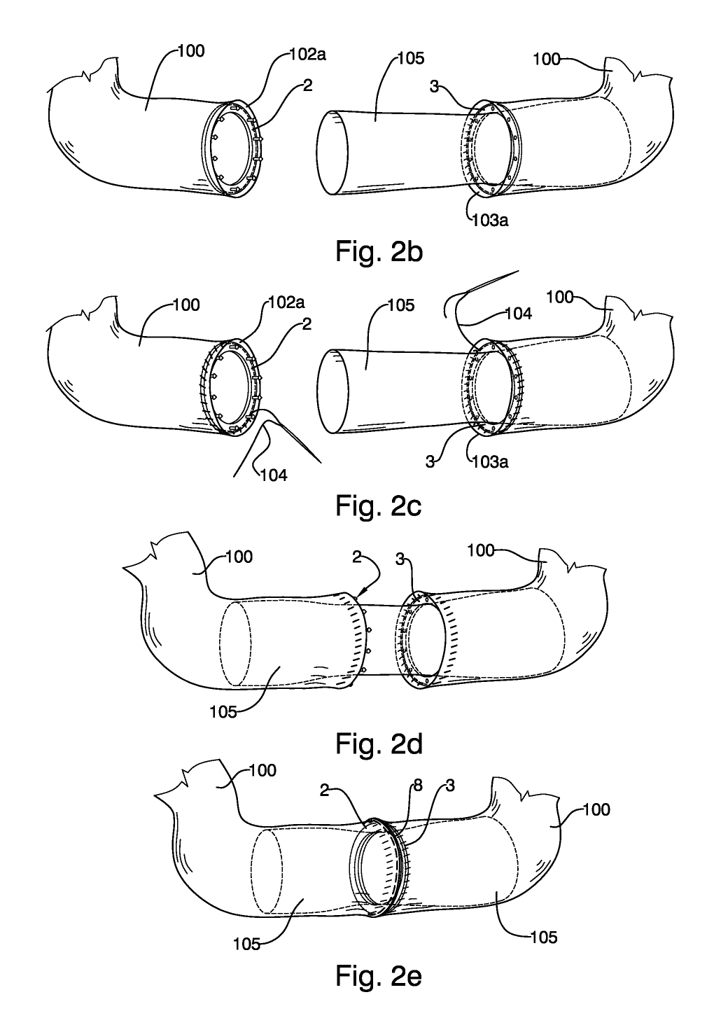

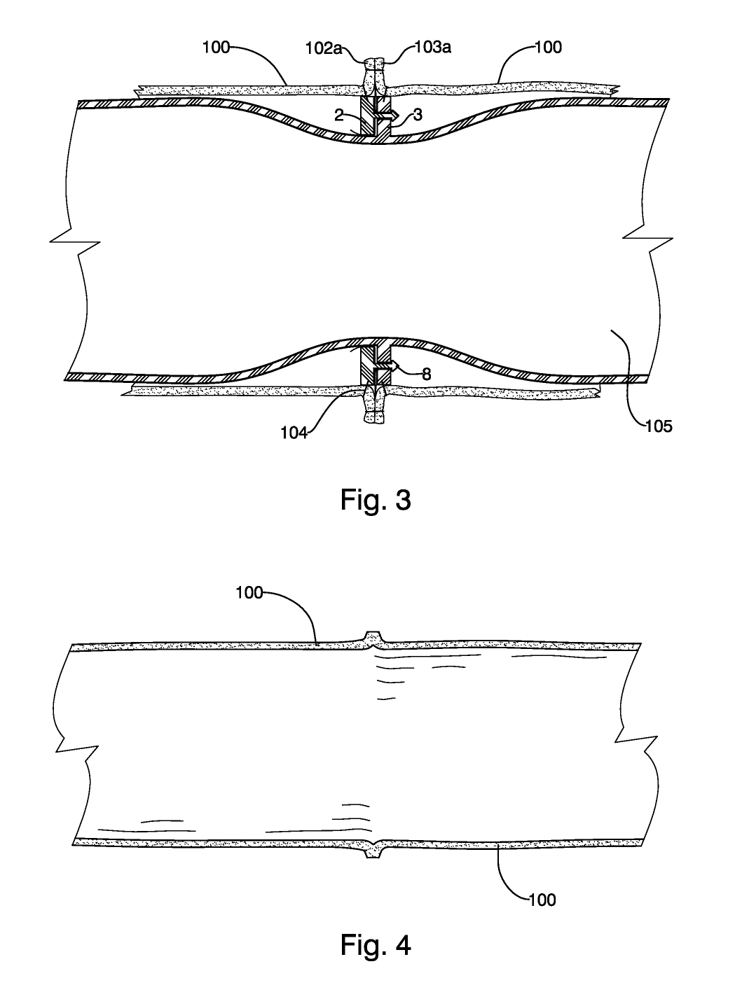

FIGS. 2A-2E show in different cross-sectional schematic views the surgical procedure proposed by the present invention. First, FIG. 2A shows a bowel 100 which is sectioned to remove a portion 101. This procedure is not illustrated in detail as the surgical procedure per se through which the bowel is cut does not form part of the present invention. Once section 101 is removed, two loose ends 102-103 must be immediately anastomosed. Each end includes an end edge or lip 102a-103a which will be approached and sutured as will be explained in detail below.

FIG. 2B illustrates the second step of the purposed surgical procedure in which the surgeon fits the first clamp member 2 into one open end 102 and the remaining member 3 together with the integrated tube 105 into the other open end of the bowel 103. Said tube 105 has enough flexibility to be easily introduced into the bowel but at the same time its consistency is firm enough to keep the walls of the bowel separated. A small annular portion of bowel tissue is left between each clamp member 2-3 and the end edge of lip 102a-103a to define a portion of tissue which may be approximated at the end of the procedure.

FIG. 2C shows the moment in which the clamp member 2 is anchored to the bowel. In order to be able to do that, said annular clamp member 2 includes a circular indentation 11 so that the bowel can be anchored in place by tying a suture around it. Said suture is schematically indicated through a curling line 104 going around the indentations 11 and the bowel's tissue. Also said clamp member 3 plus the tube 105 integral thereof are also inserted into the bowel 100 and due to the diameter of said stent-like tube 105 said integral pieces 3-105 can be kept in place without suturing to the bowel. The length of the tube must extend at least four or five centimeters to either side of the annular clamps, but different lengths and diameters are applied depending on the bowel being protected.

FIG. 2D shows the step in which the clamp member 2 is introduced into the stent-like tube 105 and is moved towards clamp 3. This is called the approximating maneuver. This approximation maneuver ends when both rings and clamp members 2-3 are one in front of the other, both around said stent-like tube 105. Spikes 8 are aligned with receptacles 9 so after the approximating maneuver each spike 8 remains in front of one corresponding receptacle 9. The advantage of the present invention resides on the possibility of anastomosing two open ends 102-103 of bowel 100 and it protects the anastomosis by also having an inner stent-like tube 105. Said rings 2-3 as well as the stent-like tube 105 come in different sizes to fit the different circumferences of the bowel, and the inner tube 105 must be rigid enough to maintain its shape while it is being placed into the bowel 100.

Finally, FIG. 2E shows the last part of the procedure in which the ends 102-103 are finally approximating by approaching the rings 2-3 with the bowel secured in place, around the stent-like tube 105. When the rings are one in front of the other, and the end edges or lips are also in touch, the spikes 8 are forced to go through receptacles 9 creating a firm link between them. Since each ring is anchored to the bowel both open ends 102-103 may be approximated using any traditional suturing device like clips, sutures, glue, or any other devices or energies.

The surgical procedure illustrated in FIGS. 2A-2E in which the approximating device of FIG. 1 is used to perform the anastomosis of bowel tissue, is especially useful for performing a fast, secure and accurate anastomosis maneuver that will minimizes or prevents dramatically the leaks for this type of procedures on this type of tissue. It is possible the surgeon will be able to perform a complete approximation procedure that demands, with the devices of the prior art, firing the clamp machine several times.

It is also important to point out said rings in all the previously described embodiments come in different sizes to fit the different circumferences of the bowel. The inner tube 105 must be rigid enough to maintain its shape while it's being placed into the bowel.

In summary, the method for anastomosing a bowel includes the following steps:
a) Cutting the bowel for removing a portion thereof, thus defining two loose ends of bowel tissue;
b) Inserting into one loose end of bowel tissue a first annular bio-absorbable clamp member, including a set of spikes on the front face with a circumferential indentation,
c) Inserting into the second open end of bowel a second annular clamp member including a stent-like tube integral therewith and set of complementary receptacles aligned with said spikes and with a circumferential indentation on its front face;
d) Anchoring said first clamp members to the bowel's tissue;
e) Introducing said first clamp member around said stent-like tube also made of bio-absorbable material;
f) Approximating both clamp members with the overlying secured bowel by moving said first clamp member towards said second clamp member around the stent-like tube until the first annular member remains in front of the second annular clamp member;
g) Inserting said spikes into said receptacles, and
h) Approximating the end edges of said open ends of the bowel if necessary.

Finally, the purposed invention can be used for primary anastomosis as was explained above but this should not be understood as a limitation of the possibilities of this invention. Even though a preferred embodiment was described in which the approximating device is delivered via an endoscope, colonoscope, or a bougie like delivery mechanism, it can also be used to protect an anastomosis created by other means (sutures, staples, glue, etc.). That is, the piece defined by the clamp member 3 and the stent-like tube 105 as a single piece may also be used to protect another anastomosis already performed by other traditional methods. By doing this, said tube 105 may protect an anastomosis from leakage and thus avoiding the risks involved therein. While the preferred embodiments of the invention have been described above, it will be recognized and understood that various modifications can be made in the invention and the appended claims are intended to cover all such modifications which may fall within the spirit and scope of the invention.

I claim:

1. Tissue approximating device, comprising a two-piece anastomosis clamp including a first member and a second clamp member; the first clamp member includes a set of spikes with a circular indentation and the second clamp member includes a stent-like tube integral therewith and a set of receptacles for receiving said spikes and also a circular indentation.

2. The tissue approximating device in accordance to claim 1, wherein both members are made of an absorbable material.

3. The tissue approximating device, in accordance to claim 1, wherein the anastomosis is performed via open technique.

4. The tissue approximating device, in accordance to claim 1, wherein the anastomosis is performed via Natural Orifice Translumenal Endoscopic Surgery (NOTES).

5. The tissue approximating device, in accordance to claim 1, wherein the anastomosis is performed via a single site surgery.

6. The tissue approximating device, in accordance to claim 1, wherein the first member and the second member have a circular shape.

7. The tissue approximating device, in accordance to claim 1, wherein each spike has an arrow-like shape.

8. The tissue approximating device, in accordance to claim 7, wherein the body of each spike has a rectilinear thin shape.

9. The tissue approximating device, in accordance to claim 8, wherein the tip of said spike has a V-shape.

10. The tissue approximating device, in accordance to claim 8, wherein the tip of said spike has a blunt rounded shape.

11. The tissue approximating device, in accordance to claim 8, wherein the tip of said spike has a one shaped shape.

12. The tissue approximating device, in accordance to claim 1, wherein said stent-like tube integral to the second clamp member is a flexible tube made also of an absorbable material.

13. The tissue approximating device, in accordance to claim 1, wherein said stent-like tube integral to the second clamp member is a flexible tube made also of a biodegradable material

14. The tissue approximating device, in accordance to claim 1, wherein the outer portion of the clamps have a circumferential indentation (so that the bowel can be anchored in place by tying a suture around the bowel in this indentation).

15. The tissue approximating device, in accordance to claim 1, wherein the spikes and the receptacle are aligned.

16. A method for anastomosing a bowel, comprising the following steps:
a) Cutting the bowel for removing a portion thereof, thus defining two loose ends of bowel tissue;
b) Inserting into one loose end of bowel tissue a first annular bio-absorbable clamp member, including a set of spikes on the front face and a circumferential indentation of the clamp member,
c) Inserting into the second loose end of bowel a second annular clamp member including a stent-like tube integral therewith and set of complementary receptacles aligned with said spikes and with a circular indentation indentations;
d) Anchoring said first clamp members to the bowel's tissue;
e) Introducing said first clamp member around said stent-like tube also made of bio-absorbable material;
f) Approximating both clamp members by moving said first clamp member towards said second clamp member around the stent-like tube until the first annular member remains in front of the second annular clamp member;
g) Inserting said spikes into said receptacles, and
h) Approximating the end edges of said loose ends of the bowel if necessary

17. The method for anastomosing a bowel, in accordance to claim 16, wherein the spikes and the indentations of said first clamp member are arranged in accordance to coaxial circles.

18. The method for anastomosing a bowel, in accordance to claim 16, wherein said first clamp member is secured to the bowel.

19. The method for anastomosing a bowel, in accordance to claim 16, wherein the end edges of said loose ends are approximated.
